(19) United States
(12) Patent Application Publication
(10) Pub. No.: US 2011/0160136 A1
(43) Pub. Date: Jun. 30, 2011

(54) POLYPHENOLS FOR THE TREATMENT OF CARTILAGE DISORDERS

(75) Inventors: Elizabeth Offord Cavin, Montreux (CH); Gary Williamson, Harrogate (GB); Didier Courtois, St-Avertin (FR); Bernard Lemaure, Tours (FR); Andre Touche, Monts (FR); Grace Ing Soon, Singapore (SG); Laurent Ameye, Lausanne (CH)

(73) Assignee: NESTEC S.A., Vevey (CH)

(21) Appl. No.: 12/919,163

(22) PCT Filed: Feb. 25, 2008
(86) PCT No.: PCT/EP08/52265

§ 371 Date: Aug. 24, 2010

(51) Int. Cl. A61K 38/02; A61P 19/02; A61K 31/235; A61P 3/02; A61K 31/366; A61K 31/352

(52) U.S. Cl. 514/16.6; 514/532; 514/17.1; 514/453; 514/456

(57) ABSTRACT

Compositions and methods for maintenance of cartilage health or prevention, alleviation and/or treatment of cartilage disorders are presented. The present invention also provides the manufacture of a nutritional product, a supplement or a medicament for promoting cartilage growth or for the maintenance of cartilage health and methods regarding same. In an embodiment, the present invention provides a composition comprising an active ingredient having an effective amount of a plant or plant extract containing at least one phytochemical, in particular polyphenol having the ability to treat or prevent muscular skeletal diseases.

The present invention generally relates to nutritional compositions that provide health benefits. More specifically the present invention relates to the use of a composition comprising at least one polyphenol for the preparation of a product to improve the condition of cartilage, in particular, to treat or prevent muscular skeletal disease. In particular the present invention relates to a use in accordance with claim 1.

Osteoarthritis (OA) is the first cause of disability in the elderly. Currently, no cure exists for osteoarthritis and therapy is only palliative aiming at improving symptoms. Current recommendations for the management of osteoarthritis include a combination of nonpharmacological interventions (weight loss, education programs, exercise, and so on) and pharmacological treatments (paracetamol, nonsteroidal anti-inflammatory drugs [NSAIDs], and so on). Among these pharmacological treatments, NSAIDs, despite serious adverseeffects associated with their long-term use, remain among the most widely prescribed drugs for OA.

The object of this invention is to provide alternative compositions from natural sources that can be used to improve the condition of cartilage in a human or animal body.

The present inventors achieved this object by providing the art with a use in accordance with claim 1.

The present invention generally relates to compositions for maintenance of cartilage health or the prevention, alleviation and/or treatment of cartilage disorders. The present invention also provides new compounds, 6″-Feruloylnepitrin, 6″-Coumaroylnepitrin and a compound

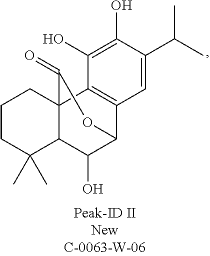

dehydroxy rosmarinic acid which may be used in the compositions of the present invention. The present invention relates to the manufacture of a food product, a beverage, a nutritional product, a supplement or a medicament for promoting cartilage growth, for decreasing cartilage degeneration or destruction, or for the maintenance of cartilage health and methods regarding same. In particular, the present invention provides the manufacture of a food product, a beverage, a nutritional product, a supplement or a medicament for promoting cartilage formation which is important for cartilage growth as well as for the maintenance of cartilage health through balanced cartilage remodeling and methods regarding same.

In an embodiment, the present invention provides the use of a composition comprising at least one polyphenol for the preparation of a product to treat or prevent muscular skeletal disease. The at least one polyphenol is preferably selected from the group consisting of dehydroxy rosmarinic acid, 6″-Feruloylnepitrin, 6″-Coumaroylnepitrin, eupafolin, carnosol, scutellarin, kaempferol, rosmarinic acid, rosmanol, cirsimaritin, luteolin, 6-methoxy-luteolin, 7-epirosmannol, or mixtures thereof, and may be provided in the form of a plant extract.

Hence, in an embodiment, the present invention provides a composition comprising an active ingredient having an effective amount of at least one polyphenol, having the ability to treat or prevent muscular skeletal disease. The polyphenol is preferably obtained from plant sources.

If a plant extract is used, it is particularly preferred that the plant extract is a rosemary and/or caraway plant extract.

The product may be a medicament, a beverage, a food product, nutritional supplement and/or nutraceutical for humans and/or pets. Preferably, the product comprises the at least one polyphenol in an amount of 0.001-100 wt.-% of the total dry weight of the composition. The at least one polyphenol may be to be administered in an amount of 0.01 μg-100 mg per kg body weight per day.

The product may further comprise a protein source, a fat source and/or a carbohydrate source. The protein source may provide about 1-55% of the total energy of the product, the fat source may provide about 5-55% of the total energy of the product, and the carbohydrate source may provide about 40-80% of the total energy of the product.

The product may be intended for oral and/or enteral application.

Preferably, the product may be in a form selected from the group consisting of a nutritionally balanced food, a nutritionally complete formula, a dietary supplement, a dairy product, a chilled or shelf stable beverage, a soup, a nutritional bar, pet food, confectionery, a pharmaceutical composition and combinations thereof.

The product prepared by the use of the present invention may be used to treat or prevent osteoarthritis, to modulate the ratio of cartilage anabolism and cartilage catabolism and/or to inhibit cartilage catabolism.

In an alternative embodiment, the present invention provides a method for manufacturing a food composition for the prevention, the alleviation and/or the treatment of cartilage disorders or maintenance of cartilage health in humans or pets, the method comprising providing a food composition; and adding to the food composition an active ingredient having a plant or a plant extract containing at least one phytochemical, in particular polyphenol, having the ability to treat or prevent muscular skeletal disorders to prepare the composition. For example, the composition can include components chosen from the group consisting of chicory, tea, cocoa, bioactives, antioxidants, fatty acids, prebiotic fibers, glucosamine, chondroitin sulphate and combinations thereof.

In another embodiment, the present invention provides a method for the treatment, alleviation or prevention of osteoarthritis and/or rheumatoid arthritis, the method comprising administering a therapeutically-effective amount of a composition comprising an active ingredient having an effective amount of at least one plant or plant extract containing at least one phytochemical, in particular polyphenol.

The composition prepared by the use of the present invention may also be used to treat or prevent the symptoms of osteoarthritis and/or rheumatoid arthritis, such as for example pain and impaired mobility.

In an alternative embodiment, the present invention provides a method to modulate the ratio of cartilage anabolism and cartilage catabolism, the method comprising feeding an individual, a composition comprising an active ingredient having an effective amount of at least one polyphenol. The polyphenol is preferably obtained from plant sources.

In another embodiment, the present invention provides a method for the treatment, alleviation and/or prophylaxis of osteoarthritis in pets and humans, the method comprising feeding an individual having or at risk of osteoarthritis, a composition comprising an active ingredient having an effective amount of at least one polyphenol, having the ability to induce bone morphogenic protein expression in the individual.

In still another embodiment, the present invention provides a method to inhibit cartilage catabolism, the method comprising administering to an individual a therapeutically effective amount of a composition comprising an active ingredient having an effective amount of at least one polyphenol.

In a further embodiment, the present invention provides a method to treat or prevent muscular skeletal disease, the method comprising feeding an individual, a composition comprising an active ingredient having an effective amount of at least one polyphenol.

Additional features and advantages of the present invention are described herein, and will be apparent from, the following detailed description, the figures and the examples.

### BRIEF DESCRIPTION OF THE FIGURES

FIG. 1 illustrates an extraction protocol.

FIG. 2 illustrates a summary of the extraction procedure and first fractionation.

FIGS. 3 and 4 illustrate compounds isolated from plant extracts

FIG. 5 Effect of carnosol on interleukin 1β-induced glycosaminoglycan release

FIG. 6 Effect of kaempferol on interleukin 1β-induced glycosaminoglycan release.

FIG. 7 Effect of scuttelarein on interleukin 1β-induced glycosaminoglycan release

FIG. 8 Effect of 6-methoxyluteolin on interleukin 1β-induced glycosaminoglycan release

The present invention relates to beneficial compositions that can be used, for example, to improve the status of cartilage and its formation and methods regarding same. For example, in an embodiment, the present invention is directed to polyphenols obtained from plants and plant extracts that stimulate cartilage formation. The polyphenols of the present invention may also be provided in the form of plant extracts.

In embodiments of the present invention, extracts comprising the polyphenols of the present invention can be prepared from edible and/or medicinal plant species and were found to have a potential to stimulate cartilage formation. As discussed in more detail later, extracts were generally prepared by a process comprising the following steps (a) hexane, (b) methanol-water, (c) methanol-water extracts hydrolyzed with glycosidases and re-extracted with ethylacetate, and (d) removal of large polyphenols with a polyvinyl polypyrrolidone (PVPP) column. The pure compounds were used for in vitro screening.

It was surprisingly found that several polyphenolic compounds extracted, e.g., from rosemary plants could be used as active compounds for cartilage development, growth and/or maintenance. For example, 6″-Feruloylnepitrin, 6″-Coumaroylnepitrin, dehydroxy rosmarinic acid, eupafolin, carnosol, scutellarin, kaempferol, rosmarinic acid, rosmanol, cirsimaritin, luteolin, 6-methoxy-luteolin and 7-epirosmannol exhibit an anti-catabolic potential.

Three constituents of the active rosemary extract are new, and have never been described in the literature. These are 6″-Feruloylnepitrin, 6″-Coumaroylnepitrin, and dehydroxy rosmarinic acid (C0063-W-06).

In an embodiment of the present invention, the MeOH/water extracts of the rosemary plant (25% of the initial leaf dry matter) obtained after a defatting step with hexane contain the molecules responsible for the activity and could be used in a food product. If plant extracts are used, it is preferred if these plant extracts are enriched in the polyphenols of the present invention. It is preferred if the concentration of the polyphenols of the present invention in the enriched plant extract is at least 2 fold, preferably 10 fold, even more preferred at least 50-fold compared to their content in the MeOH/water extracts described above. Compared to the natural plant the content of the polyphenols of the present invention in the enriched extract is preferably at least 10 fold increased, preferably at least 100 fold increased, even more preferred at least 500-fold increased.

The polyphenols of the present invention as well as plants or plant extracts comprising them may be used in the preparation of a food composition. The composition may be in the form of a nutritionally balanced food or pet food, a dietary supplement, a treat or a pharmaceutical composition.

The polyphenols of the present invention and/or plants or plant extracts comprising them may be used alone or in association with other plants such as chicory, tea, cocoa, or with other bioactive molecule such as antioxidants, fatty acids, prebiotic fibers, glucosamine, chondroitin sulphate, for example.

In one embodiment of the present invention, a food composition or nutritional formula for human consumption is prepared. This composition may be a nutritional complete formula, a dairy product, a chilled or shelf stable beverage, soup, a dietary supplement, a meal replacement, and a nutritional bar or a confectionery.

Apart from the polyphenols of the present invention or plants or plant extracts comprising them, in particular rosemary plants or plant extracts, the nutritional formula may comprise a source of protein. Dietary proteins are preferably used as a source of protein. The dietary proteins may be any suitable dietary protein; for example animal proteins (such as milk proteins, meat proteins and egg proteins); vegetable proteins (such as soy protein, wheat protein, rice protein, and pea protein); mixtures of free amino acids; or combinations thereof. Milk proteins such as casein, whey proteins and soy proteins are particularly preferred. The composition may also contain a source of carbohydrates and a source of fat.

If the nutritional formula includes a fat source, the fat source preferably provides about 5% to about 55% of the energy of the nutritional formula; for example about 20% to about 50% of the energy. The lipids making up the fat source may be any suitable fat or fat mixtures. Vegetable fats are particularly suitable; for example soy oil, palm oil, coconut oil, safflower oil, sunflower oil, corn oil, canola oil, lecithins, and the like. Animal fats such as milk fats may also be added if desired.

A source of carbohydrate may be added to the nutritional formula. It preferably provides about 40% to about 80% of the energy of the nutritional composition. Any suitable carbohydrates may be used, for example sucrose, lactose, glucose, fructose, corn syrup solids, and maltodextrins, and mixtures thereof. Dietary fiber may also be added if desired. If used, it preferably comprises up to about 5% of the energy of the nutritional formula. The dietary fiber may be from any suitable origin, including for example soy, pea, oat, pectin, guar gum, gum arabic, and fructooligosaccharides. Suitable vitamins and minerals may be included in the nutritional formula in an amount to meet the appropriate guidelines.

One or more food grade emulsifiers may be incorporated into the nutritional formula if desired; for example diacetyl tartaric acid esters of mono- and di-glycerides, lecithin and mono- and di-glycerides. Similarly suitable salts and stabilizers may be included. Vitamins and minerals may also be combined with the plant extract.

The nutritional composition is preferably enterally administrable; for example in the form of a powder, tablet, capsule, a liquid concentrate, solid product or a ready-to-drink beverage. If it is desired to produce a powdered nutritional formula, the homogenized mixture is transferred to a suitable drying apparatus such as a spray drier or freeze drier and converted to powder.

In another embodiment, a nutritional composition comprises a milk-based cereal together with a prebiotic formulation. Preferably the nutritional composition is intended for adults, in particular middle-aged or elderly people.

In another embodiment, a usual food product may be enriched with at least one plant or plant extract according to the present invention. For example, a fermented milk, a yoghurt, a fresh cheese, a renneted milk, article of confectionery, for example a sweet or sweetened beverage, a confectionery bar, breakfast cereal flakes or bars, drinks, milk powders, soy-based products, non-milk fermented products or nutritional supplements for clinical nutrition.

The amount of the new compounds and/or polyphenols of the present invention, and—if present—plant or plant extract in the composition may vary according to its source and its utilization. In a preferred embodiment, an efficient daily dose amount is of at least about 1 mg, and more preferably from 1 mg to 10 g of the active molecule per day.

In an alternative embodiment, a pharmaceutical composition containing at least polyphenol as described above, in an amount sufficient to achieve the desired effect in an individual can be prepared. This composition may be a tablet, a liquid, capsules, soft capsules, pastes or pastilles, gums, or drinkable solutions or emulsions a dried oral supplement, a wet oral supplement. The pharmaceutical composition can further contain carriers and excipients that are suitable for delivering the respective active molecule of different nature to the target tissue. The kind of the carrier/excipient and the amount thereof will depend on the nature of the substance and the mode of drug delivery and/or administration contemplated. It will be appreciated that the skilled person will, based on his own knowledge select the appropriate components and galenic form.

The polyphenol of the present invention may be used in the preparation of a pet food composition. The said composition may be administered to the pet as a supplement to its normal diet or as a component of a nutritionally complete pet food, and more preferably in an hypocaloric pet food. It may also be a pharmaceutical composition.

The polyphenol of the present invention may be used alone or in association with other plants such as chicory, tea, cocoa, or with other bioactive molecule such as antioxidants, fatty acids, prebiotic fibers, glucosamine, chondroitin sulphate for example.

Preferably, a pet food composition prepared in accordance with the present invention contains about 0.01 to 100 mg of the compounds and/or polyphenols per gram of dry pet food. The nutritionally complete pet food composition according to the invention may be in powdered, dried form, a treat or a wet, chilled or shelf stable pet food product. It may be chilled or provided as a shelf stable product. These pet foods may be produced by ways known in the art.

The pet food may optionally also contain a prebiotic, a probiotic microorganism or another active agent, for example a long chain fatty acid. The amount of prebiotic in the pet food is preferably less than 10% by weight. For example, the prebiotic may comprise about 0.1% to about 5% by weight of the pet food. For pet foods which use chicory as the source of the prebiotic, the chicory may be included to comprise about 0.5% to about 10% by weight of the feed mixture; more preferably about 1% to about 5% by weight.

If a probiotic micro-organism is used, the pet food preferably contains about 10⁴ to about 10¹⁰ cells of the probiotic micro-organism per gram of the pet food; more preferably about 10⁶ to about 10⁶ cells of the probiotic micro-organism per gram. The pet food may contain about 0.5% to about 20% by weight of the mixture of the probiotic micro-organism; preferably about 1% to about 6% by weight; for example about 3% to about 6% by weight.

If necessary, the pet food can be supplemented with minerals and vitamins so that they are nutritionally complete. Further, various other ingredients, for example, sugar, salt, spices, seasonings, flavoring agents, and the like may also be incorporated into the pet food as desired.

In another embodiment, dietary adjuncts may be prepared so as to improve pet food quality. As dietary adjuncts, they may be encapsulated or may be provided in powder form and packaged in conjunction with or separately from a main meal, be it wet or dry. By way of example, a powder containing extracts according to the invention, may be packed in sachets in a powder form or in a gel or lipid or other suitable carrier. These separately packaged units may be provided together with a main meal or in multi-unit packs for use with a main meal or treat, according to user instructions.

Administering to a human or animal, the food or pet food composition as described above, can result in an improved cartilage regeneration. It can help to stimulate cartilage formation. In particular, it may provide an optimal cartilage generation during childhood. This food composition can help to prevent cartilage loss, in particular cartilage loss associated with age in mammals or cartilage loss associated with long term hospitalization. Furthermore, it can help to build cartilage in mammals and prevent osteoarthritis in pets and humans, which results in a better activity or mobility of the individual (e.g. pets and/or humans).

It is clear to those of skill in the art that they can freely combine features described in this application. Further advantages and features of the present invention will be apparent from the following figures and examples.

### EXAMPLES

By way of example and not limitation, the following examples are illustrative of various embodiments of the present invention and further illustrate experimental testing conducted in accordance with embodiments of the present invention.

### Example 1

### Plant Polyphenols

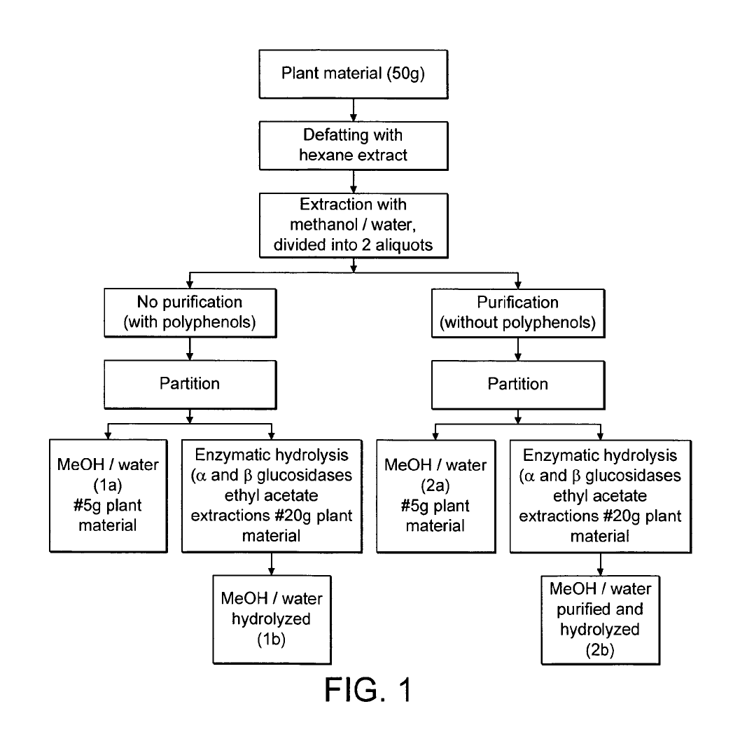

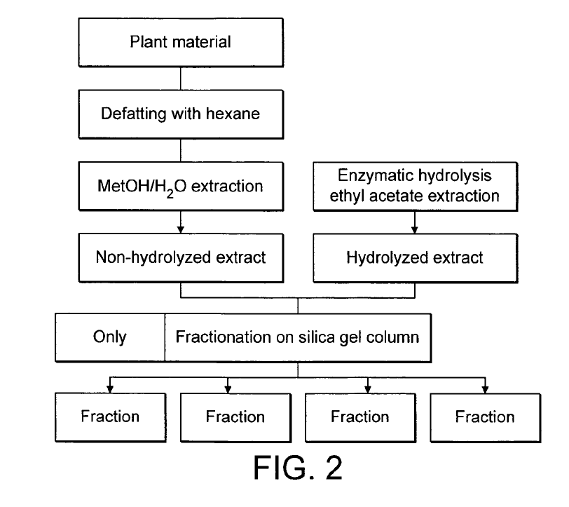

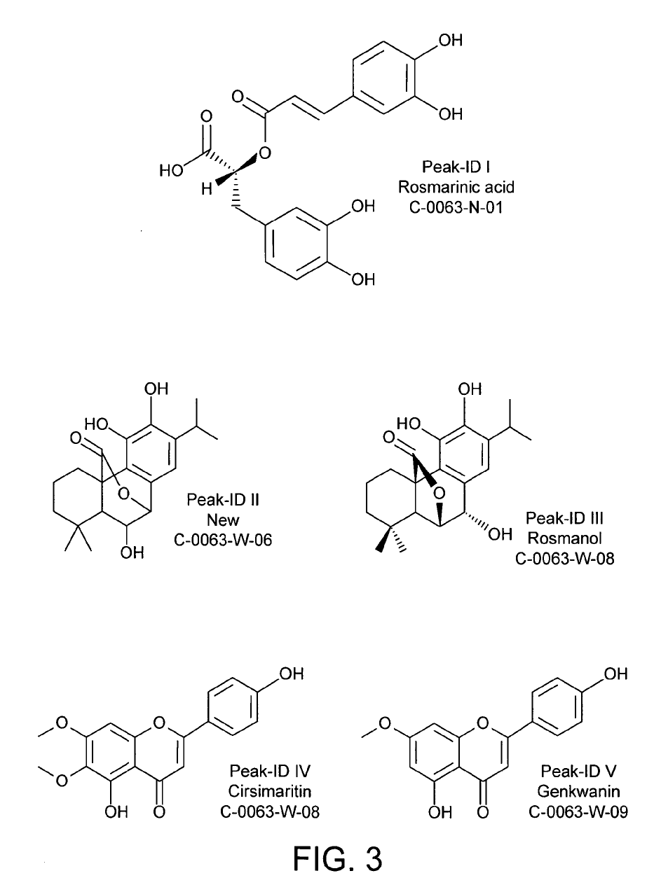

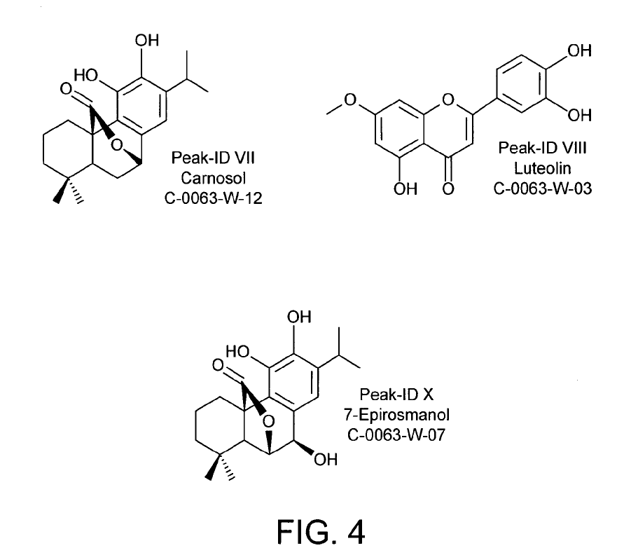

The following compounds were purified from at least one plant source, such as soybean, sweet flag, service berry, mugwort/wormwood, nutgrass, dandelion, spice bush, peach, sweet iris, rosemary, caraway, thyme, spearmint, grape and chicory. FIGS. 1 and 2 illustrate a typical extraction protocol. FIGS. 3 and 4 show some compounds isolated accordingly.

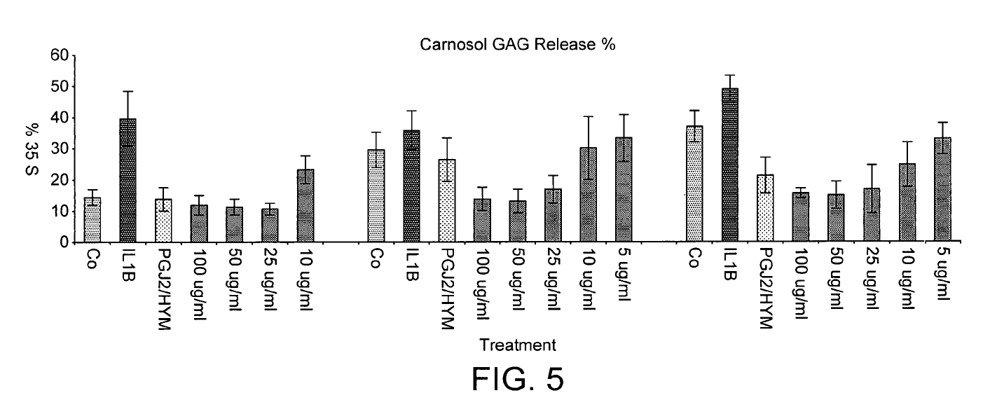

To demonstrate the claimed effects of the polyphenols of the present invention several isolated and purified plant polyphenols were positively tested for their effects on IL1 β-induced GAG release (FIGS. 5-8).

The experiments were conducted as follows:

Articular cartilage explants are dissected out of the metacarpophalangeal joint of old cows (8-10 years). The skin is removed from the feet. The articulation is opened transversally. Intra articular ligaments are transected. Full thickness slices of cartilage are dissected out and put in a Petri dish containing DMEM supplemented with 20% FBS (fetal bovine serum albumin) and antibiotics (penicillin, streptomycin and gentamycin). Under the hood, disks of cartilage obtained by using a biopsy punch (6 mm in diameter) are distributed between the wells of a 96-wells plate containing 200 μl of medium (DMEM+20% FBS, 1% penicillin/streptomycin, 0.1% gentamycin) per well. Finally, plates are put into an incubator (37° C./5% CO2).

Five days after their harvest, the explants are divided into several different treatment conditions:

1—negative control: the wells contain only culture medium.
2—positive stimulatory control: Interleukin 1β(IL1β) is added to the media at the concentration of 50 ng per ml. This cytokine is used to stimulate the catabolism and to induce inflammation.
3—positive inhibitory control: d15-PGJ2 (a peroxisome proliferator activated receptor γ agonist) and hymenialdisine are co-added, to the medium, with IL1β, to counteract the catabolic effect of this cytokine (Sabatini M, et al. (2002) Osteoarthritis Cartilage 10(9): 673-679; Scher J U, et al. (2005) Clin Immunol 114(2): 100-109; Boyault S, et al. (2001) Febs Letters 501(1): 24-30)
4, 5, 6, 7, 8 (if present)—tested compounds: these conditions test the effects of three concentrations of a polyphenol in presence of IL1β.

The explants undergo these treatments for 72 hours in the incubator (37° C., 5% CO₂). The following protocol is based on the disclosure of Campbell et al. (1984) Arch Biochem Biophys 234(1): 275-289 and Hascall V C, et al. (1983) ArchBiochem Biophys 224(1): 206-223 and was carried out 48 hours after the harvest of the explants,

Sulphur 35 (35S) was added to the medium to be incorporated into newly synthesized polysulfated glycosaminoglycans within the extracellular matrix of the explants. After 72 hours, several washes were performed to eliminate the non-incorporated radioactivity. The explants were then treated for 72 hours, after which the supernatants were collected and the amount of radioactivity released in the medium was measured with a beta counter, as a marker of glycosaminoglycan (GAG) catabolism. The radioactivity present in the explants was also measured and used to normalize the amount of radioactivity released in the media. The radioactivity was expressed in DPM (degradations per minute) and the GAG release was assessed by the following formula:

GAG release=radioactivity in the supernatants/(radioactivity in the supernatants+radioactivity in the explants)

### Example 2

### Effect of Carnosol on IL1 β-Induced GAG Release

After being loaded with radioactivity, cartilage explants were treated for 72 hours with DMEM only (in blue) or with 50 ng/ml of IL1β only (in red) or with IL1β and PGJ2 and hymenialdisine (in yellow), a positive control of inhibition, or with IL1β and carnosol (in light pink). The dose range for carnosol was: 100-50-25-10-5 μg/ml. Data are expressed as the percentage of radioactivity release (mean of 6 culture wells±standard deviation), with percentage of radioactivity release=supernatant radioactivity/(supernatant radioactivity+explants radioactivity). The percentage of radioactivity released is used as a surrogate marker for GAG release. Three independent experiments are represented. The results are shown in FIG. 5.

### Example 3

### Effect of Kaempferol on IL1 β-Induced GAG Release

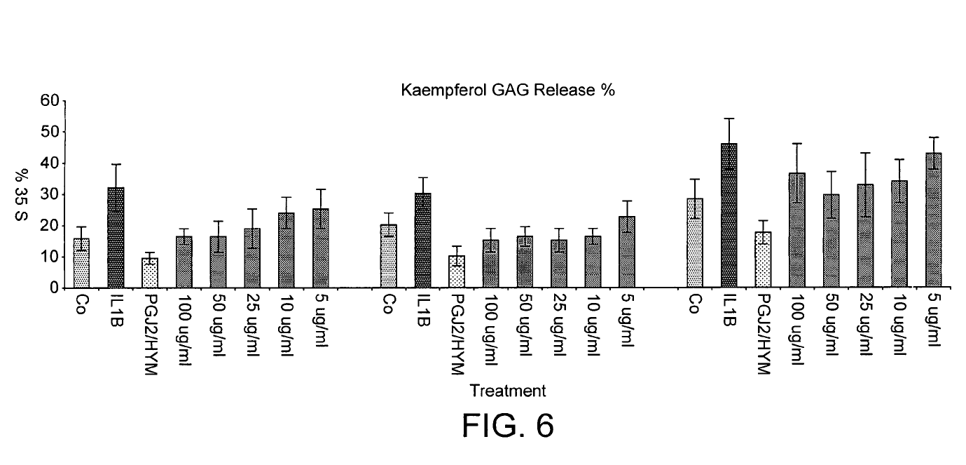

After being loaded with radioactivity, cartilage explants were treated for 72 hours with DMEM only (in light blue) or with 50 ng/ml of IL1β only (in red) or with IL1β and PGJ2 and hymenialdisine (in yellow), a positive control of inhibition, or with IL1β and kaempferol (in dark blue). The dose range for kaempferol was: 100-50-25-10-5 μg/ml. Data are expressed as the percentage of radioactivity release (mean of 6 culture wells±standard deviation), with percentage of radioactivity release=supernatant radioactivity/(supernatant radioactivity+explants radioactivity). The percentage of radioactivity released is used as a surrogate marker for GAG release. Three independent experiments are represented. The results are shown in FIG. 6.

### Example 4

### Effect of Scuttelarein on IL1 β-Induced GAG Release

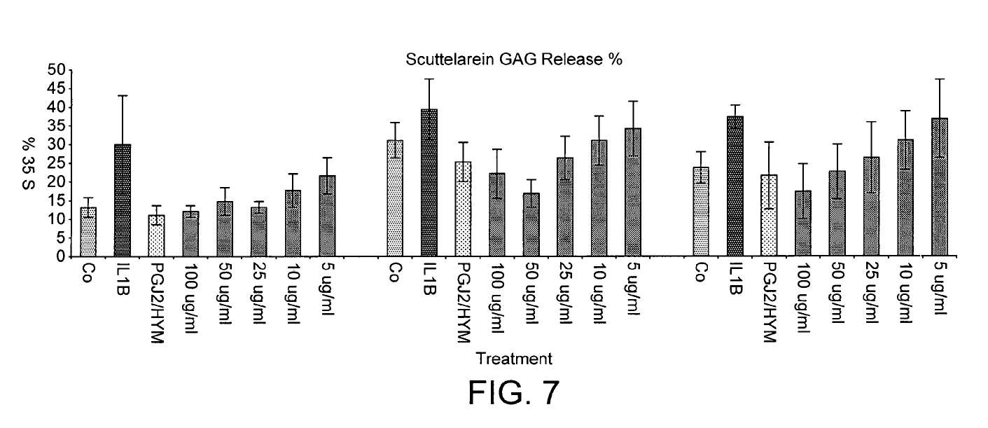

After being loaded with radioactivity, cartilage explants were treated for 72 hours with DMEM only (in light blue) or with 50 ng/ml of IL1β only (in red) or with IL1β and PGJ2 and hymenialdisine (in dark yellow), a positive control of inhibition, or with IL1β and scuttelarein (in light yellow). The dose range for scuttelarein was: 100-50-25-10-5 μg/ml. Data are expressed as the percentage of radioactivity release (mean of 6 culture wells±standard deviation), with percentage of radioactivity release=supernatant radioactivity/(supernatant radioactivity+explants radioactivity). The percentage of radioactivity released is used as a surrogate marker for GAG release. Three independent experiments are represented. The results are shown in FIG. 7.

### Example 5

### Effect of 6-methoxyluteolin on IL1 β-Induced GAG Release

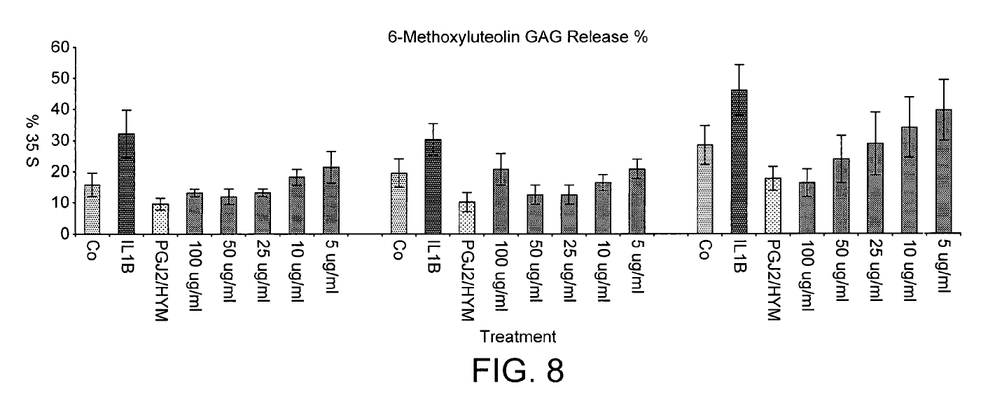

After being loaded with radioactivity, cartilage explants were treated for 72 hours with DMEM only (in light blue) or with 50 ng/ml of IL1β only (in red) or with IL1β and PGJ2 and hymenialdisine (in yellow), a positive control of inhibition, or with IL1β and 6-methoxyluteolin (in light orange). The dose range for 6-methoxyluteolin was: 100-50-25-10-5 μg/ml. Data are expressed as the percentage of radioactivity release (mean of 6 culture wells±standard deviation), with percentage of radioactivity release=supernatant radioactivity/(supernatant radioactivity+explants radioactivity). The percentage of radioactivity released is used as a surrogate marker for GAG release. Three independent experiments are represented. The results are shown in FIG. 8.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method for treating or preventing muscular skeletal disease in an individual having or at risk of same comprising administering a product comprising at least one polyphenol.

2. Method in accordance with claim 1 wherein the polyphenol is provided in the form of a plant extract.

3. Method in accordance with claim 1, wherein the polyphenol is selected from the group consisting of 6″-Feruloylnepitrin, 6″-Coumaroylnepitrin and a compound with the following formula 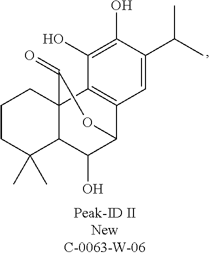
dehydroxy rosmarinic acid, eupafolin, carnosol, scutellarin, kaempferol, rosmarinic acid, rosmanol, cirsimaritin, luteolin, 6-methoxy-luteolin, 7-epirosmannol, and mixtures thereof.

4. Method in accordance with claim 2, wherein the plant extract is selected from the group consisting of a rosemary and caraway plant extract.

5. Method in accordance with claim 1, wherein the product is a medicament.

6. Method in accordance with claim 1, wherein the product is selected from the group consisting of a food product, nutritional supplement and nutraceutical for humans and/or pets.

7. Method in accordance with claim 1 wherein the disease is selected from the group consisting of osteoarthritis and rheumatoid arthritis.

8. Method in accordance with claim 1 wherein the product modulates the ratio of cartilage anabolism and cartilage catabolism.

9. Method in accordance with claim 1 wherein the individual requires an inhabitation of cartilage catabolism.

10. Method in accordance with claim 1 wherein the product comprises at least one polyphenol in an amount of 0.001-100 wt.-% of the total dry weight of the composition.

11. Method in accordance with claim 1 wherein the polyphenol is to be administered in an amount of 0.01 μg-100 mg per kg body weight per day.

12. Method in accordance with claim 1 wherein the product further comprises a protein source, a fat source and a carbohydrate source.

13. Method in accordance with claim 12 wherein the protein source provides about 1-55% of the total energy of the product, the fat source provides about 5-55% of the total energy of the product, and the carbohydrate source provides about 40-80% of the total energy of the product.

14. Method in accordance with claim 1 wherein the product is administered either orally or enterally.

15. Method in accordance with claim 1 wherein the product is in a form selected from the group consisting of a nutritionally balanced food, a nutritionally complete formula, a dairy product, a chilled or shelf stable beverage, a soup, a nutritional bar, pet food, confectionery, a pharmaceutical composition and combinations thereof.
